(19) United States
(12) Patent Application Publication
(10) Pub. No.: US 2020/0387336 A1
(43) Pub. Date: Dec. 10, 2020

(54) DISPLAY SYSTEM AND PROCESS FOR DISPLAYING AN OUTPUT OF AN ELECTROMEDICAL DEVICE

(71) Applicant: Drägerwerk AG & Co. KGaA, Lübeck (DE)

(72) Inventors: Stefan SCHLICHTING, Lübeck (DE); Jan FROST, Lübeck (DE); Maximilian MERKEL, Lübeck (DE); Christian BRANDT, Lübeck (DE); Sina SCHEUPLEIN, Lübeck (DE)

(21) Appl. No.: 16/891,805

(22) Filed: Jun. 3, 2020

(30) Foreign Application Priority Data
Jun. 7, 2019 (DE) 10 2019 003 995.5

(51) Int. Cl. G06F 3/14; G16H 15/00; G06F 21/31

(52) U.S. Cl. CPC G06F 3/14; G06F 21/31; G16H 15/00

(57) ABSTRACT

A display system (100) displays an output of an electromedical device (105). A display unit (110) displays a visual output (114) based on a received display signal (112). A display signal unit (120) receives electromedical device data (107), assigns these data to a visualization type, dynamically determines a display type depending on the visualization types to be displayed and generates the display signal based on the data and the determined display type and outputs to the display unit. A display signal monitoring unit (130) is signal connected to the display unit and the display signal unit and receives the display signal and a display unit output signal (118). The display signal contains information on the visual output carried out. The display signal monitoring unit compares the display signal and the output signal and outputs a warning signal (134) in case of a lack of agreement.

### CROSS REFERENCE TO RELATED APPLICATIONS

This application claims the benefit of priority under 35 U.S.C. § 119 of German Application 10 2019 003 995.5, filed Jun. 7, 2019, the contents of which are incorporated herein by reference.

### TECHNICAL FIELD

The invention pertains to a display system for displaying an output of at least one electromedical device and to a corresponding process for displaying an output of at least one electromedical device. The present invention further pertains to a program with a program code for carrying out the process according to the present invention.

### TECHNICAL BACKGROUND

The use of a display unit, which displays the data of a connected electromedical device and is synchronized with the mode of operation of this electromedical device, is known in the medical sector.

U.S. Pat. No. 9,104,789 B2 teaches that a display unit receives data from different connected electromedical devices and displays them in a variable manner. Pieces of alarm information are displayed here in a separate, expanded window of a control menu of the display unit.

### SUMMARY

An object of the present invention is to provide an improved output of an electromedical device, especially an especially reliable and accurately detectable output of data of at least one electromedical device.

A display system for displaying an output of at least one electromedical device with a display unit, with a display signal unit and with a display signal monitoring unit is proposed according to the present invention for accomplishing this object.

The display unit is configured to receive a display signal and to display a visual output based on the display signal. The visual output is preferably carried out via a display of the display unit.

The display signal unit has a communication interface for communication with the at least one electromedical device and is configured to receive data, which pertain to the at least one electromedical device, via the communication interface. Data that pertain to a plurality of electromedical device, for example, the electromedical devices of a common communication network, may preferably be received by the communication interface.

The display signal unit is configured here to assign these data to a visualization type, to dynamically determine a particular display type of the received data depending on the visualization types that are currently to be displayed and to generate the display signal based on the data and the respective determined display type and to output same to the display unit. The determined display type of data from a predefined subset of especially relevant data types comprises here at least as a display property the fact that a display is carried out as a visual output detectable for a user. The determined display type further comprises, as a display property, a display position within the visual output of the display unit. The display position may describe a fixed position within the visual output or it may describe a relative position, which fixes an arrangement of a display of the corresponding data relative to other data to be outputted within the framework of the visual output. The detectable visual output ensures that the display will not fail to take place in view to other data to be displayed or that it does not require an additional interaction between the user and the display unit to detect the visual output.

The display signal monitoring unit is connected to the display unit and to the display signal unit for signal technology and is configured to receive the display signal, especially to receive it from the display signal unit, and to receive an output signal from the display unit, wherein the output signal contains pieces of information on the visual output carried out. The display signal monitoring unit is configured here to compare the display signal and the output signal to one another and to output a warning signal, especially a warning signal to the display signal unit in case of a lack of agreement between the visual output carried out and the visual output to be carried out according to the display signal.

It was found within the framework of the present invention that it must be ensured when a display system that is a separate display system relative to the electromedical device that the received data are outputted visually in a suitable manner. It was found in this connection, in particular, that certain, especially relevant data types should be outputted in any case. A classification of the received data into different visualization types is used for this purpose. Based on the visualization types to be currently visualized, a respective display type can be dynamically determined hereby.

The visual output can advantageously be adapted hereby to the currently received data. In particular, especially relevant data can be markedly highlighted relative to less relevant data, for example, by a corresponding selection of a font size of the display and/or of a font type of the display and/or of the display position.

Further, it is ensured by the visual output being checked by the display signal monitoring unit that an incorrect or poorly detectable visual output, for example, based on system errors, is noticed by the display system. Due to the output of the warning signal, the display system can respond correspondingly and rearrange the visual output or at least alert the user of the display system to an error.

The display system especially advantageously ensures compatibility with different electromedical devices, without a complicated coordination having to be carried out between the display unit and a respective electromedical device. Further, the display system according to the present invention especially advantageously makes possible the simultaneous visual output of data of a plurality of electromedical devices.

Another advantage of the display system according to the present invention is that it can be ensured that the output of data is not hidden by another output, for example, by an alarm signal.

The individual units of the display system according to the present invention may be arranged each at a spaced location from one another or within a common housing. The processing of the data may preferably be carried out in this case by a common processor. As a result, the use of a plurality of processors is avoided. It is also possible, however, to carry out the functionality of the display signal monitoring unit by a unit that is separate from the display signal unit, i.e., at least by a separate processor.

The display system according to the present invention may be arranged at a spaced location from the electromedical device.

The warning signal is according to the present invention a signal that indicates a corresponding lack of agreement.

The reception of the display signal by the display unit does not rule out within the framework of the present invention that additional signals are additionally received by the display unit.

Further, the display system according to the present invention makes it advantageously possible to detect an overlap if data of another signal, for example, of an administrative display signal, are also displayed by the display unit in addition to the display signal and data of the display signal are not partially displayed or are displayed incorrectly as a result.

The dynamic determination of the respective display type comprises a determination of the display type as a function of the assigned visualization types corresponding to a currently existing number of visualization types. The dynamic determination is thus always carried out anew after the reception of data and after a corresponding assignment of a visualization type. As a result, there typically is no fixed display location for a certain visualization type, because this is determined likewise dynamically within the framework of the dynamically selected display type.

A lack of agreement is determined by the display signal monitoring unit if the visual output carried out drops below a predefined level of agreement. Thus, there is, for example, a lack of agreement if data from the predefined subset of especially relevant data types are not displayed in the visual display detectable for the user. It is advantageously ensured thereby that especially relevant data are not covered by administrative data, for example, a logon screen, a user ID and/or a device ID. The predefined level of agreement may also relate to a visual similarity in addition to the contents of the data, for example, in case of a comparison of the similarity of a font type to be displayed and a displayed font type, or to the detectability of displayed data, for example, by an assessment of an existing lack of contrast between a display color and a background color.

All the display properties combined form the respective display type of received data.

The received data are, for example, physiological parameters, which are determined by the electromedical device and/or a setting of the electromedical device, for example, a current mode of operation, an alarm limit, a configuration and/or a current patient profile, and/or an alarm, which was determined by the electromedical device.

Preferred embodiments of the display system according to the present invention will be described below.

A lack of agreement between a visual output carried out and a visual output to be carried out is present in one embodiment if relevant clinical data are not displayed or are displayed only partially within the framework of the visual output due to an overlapped display of additional data. In another embodiment, it is ensured by this comparison of a visual output carried out and of a visual output to be carried out that displayed data and especially displayed symbols can be distinguished from other data, e.g., from another number, from another letter or from another symbol.

The visual outputting is preferably carried out via a display of the display unit. In an especially preferred variant, the display is a touch display. The display is preferably enclosed by a housing of the display unit.

The display signal monitoring unit is preferably configured to output the warning signal to the display signal unit. An error in the display of the data by the display unit can be eliminated as a result directly by the display signal unit. In a variant of this embodiment, the display signal unit is configured to output an error signal to a user of the display system if the error indicated by the warning signal cannot be eliminated. Such an output to the user may be brought about, for example, by a display of an error message via the display unit.

In one embodiment, the display signal unit is further configured to carry out an adaptation of the respective, dynamically determined display type depending on the received warning signal. It can be ensured hereby that an agreement will again be reached between the visual output and the visual output to be carried out.

In an alternative and/or additional embodiment to the above embodiment, the warning signal triggers an error message which can be outputted by the display unit via the display signal. As a result, a user of the display system according to the present invention is informed that there currently is an error in the display system. The visual outputting of the error message is carried out, for example, by a change in the display type, which is visible to a user, for example, by graying out a certain area of the visual output.

In a preferred embodiment, the predefined subset of especially relevant data types comprises at least pieces of alarm information and/or clinical measured values. It is ensured hereby that at least such especially relevant data will indeed be visually displayed. This is especially advantageous when a large number of data are received by the display system, so that it is not possible to output all data simultaneously. The assigned visualization type already indicates prioritization information, which indicates against which other data a prioritization of the data of this visualization type shall be carried out. The pieces of prioritization information may be a relevance level, which indicates by a comparison with the relevance level of other data whether or not these data are prioritized compared to the other data.

In another advantageous embodiment, the display signal unit is further configured to generate the display signal such that a corresponding indication of the visual output, especially the graphic output, is displayed if it is not possible based on the dimensioning of the visual output, especially of the graphic output, to visually output all the data received from the electromedical device by the display unit. A user of the display system according to the present invention is informed hereby that not all received data are displayed via the display unit. In a preferred variant of this embodiment, non-displayed data may be displayed by an interaction between the user and the display unit. Such an interaction is, for example, the touching of a corresponding control surface on a touch display of the display unit. In another example of this variant, such an interaction is the use of a scroll bar.

In another advantageous embodiment, the display signal unit is further configured to determine a display size and/or the display position of received data of a visualization type as a function of a relevance level assigned to this visualization type. Especially relevant pieces of information are advantageously displayed in this embodiment differently than pieces of less relevant information. In particular, especially relevant pieces of information can be displayed by an especially central position within the visual output. The number of displayed data can be increased by the use of different display sizes. In particular, pieces of less relevant information can be displayed by a smaller font size than pieces of relevant information. As a result, a large number of less relevant information can be displayed simultaneously, without the display of relevant information being compromised thereby. In an alternative and/or additional embodiment, the display type comprises as a display property a display style in addition to the display size and the display position. The display style indicates, for example, a font type to be used and/or a font color to be used for the information to be displayed. Information is defined here and below as the information indicated by the respective received data.

In a preferred embodiment, the display system has, furthermore, an input unit, which is connected to the display signal unit and has a user interface. The input unit is configured here to output an input signal via the user interface depending on a user input of a user of the display system. Further, the display signal unit is configured to receive the input signal and to determine a corresponding display type depending on the visualization type assigned to the input signal and to generate and to output a display signal based on the currently received data and the respective determined display type. The user input may have, for example, authorization information, e.g., a password. Furthermore, the user input may have information pertaining to the received data, such as person-specific information. The user interface may be a keyboard, a computer mouse, a touch operating element or a joystick.

In an especially preferred embodiment, the display signal monitoring unit is further configured to output a visual signature changing at time intervals via a signature signal to the display signal unit. The display signal unit is further configured here to output the current visual signature via the display signal, and the display signal monitoring unit is further configured to compare the currently displayed visual signature to the current visual signature via the output signal and to provide checking information depending on this comparison. The ability of the display signal unit to function is advantageously checked in this embodiment. Thus, an agreement between the currently displayed visual signature and the visual signature to be displayed, which is outputted to the display signal unit, shows that the display signal unit can process received data. It is advantageously ensured hereby especially in case of use in the medical sector that newly received data, which possibly show a change in measured values of an electromedical device, continue to be processed. It can be ensured by the use of the changing visual signature that an error in the processing of data is detected especially rapidly. In an advantageous variant of this embodiment, the time intervals are regular time intervals, especially time intervals of less than 30 sec, preferably less than 20 sec and especially preferably less than 10 sec.

The display unit is configured, furthermore, in another preferred embodiment to receive an additional administrative display signal and to display a combined visual output based on the display signal and the additional administrative display signal, the additional administrative display signal indicating non-medical data of an external device. The display of the non-medical data can prevent or at least influence here the display of the data of the display signal. The display signal monitoring unit advantageously ensures in this exemplary embodiment that the data are always displayed corresponding to their assigned visualization type when they belong to the predefined subset of especially relevant data types. For example, in the case of an overlapping of these data by the non-medical data, the display signal monitoring unit would thus determine a lack of agreement between a visual output carried out and the visual output to be carried out and output a corresponding warning signal, for example. Non-medical data may be, for example, patient information received separately via the administrative display signal, a device ID, a user ID and/or a logon screen for entering a user ID.

In an especially preferred embodiment of the display system according to the present invention, the communication interface is configured to receive data from a plurality of electromedical devices. The visualization type assigned here to the data by the display signal unit preferably comprises here device information, which indicates the electromedical device corresponding to the data. The fact that the display system according to the present invention functions separately from the electromedical device, whose output shall be provided by the display system, is utilized especially advantageously in this embodiment. In a variant of this embodiment, the visualization type assigned to these data comprises a relevance level, which depends on the electromedical device corresponding to these data.

The display signal unit preferably receives via the communication interface all data of electromedical devices within an electromedical communication network. Such an electromedical communication network is assigned to the respective display system and typically comprises a plurality of electromedical devices. In a first example, the electromedical communication network comprises all electromedical devices that are used for the treatment of a single concrete patient. In a second example, the electromedical communication network comprises all electromedical devices of a single device type, which are used in a hospital ward.

The communication interface of the display signal unit according to the present invention preferably makes possible a wireless transmission of data. In an alternative and/or additional embodiment, the communication interface makes possible a cable-based transmission of data. In another embodiment, the communication interface makes possible both a wireless transmission of data and a cable-based transmission of data.

In another embodiment, the display unit is configured such that it is located at a spaced location from the display signal unit and from the display signal monitoring unit. In a variant of this embodiment, the display signal unit and the display signal monitoring unit are arranged in a common housing and are connected to the display unit for signal technology.

According to another aspect of the present invention, the above-mentioned object is accomplished by a process for displaying an output of at least one electromedical device.

The process according to the present invention has the following steps:
reception of data that pertain to the at least one electromedical device;
assignment of the received data to a respective visualization type and dynamic determination of a respective display type of the received data based on the visualization types to be currently displayed;
generation and outputting of a display signal based on the received data and of the respective determined display type, wherein the determined display type of data from a predefined subset of especially relevant data types comprises at least as the display property the fact that a display is carried out as a visual output visible to a user, and wherein the determined display type further comprises as a display property a display position;
reception of the display signal and display of a visual output based on the display signal;
transmission of an output signal, which contains information on the visual output carried out;
reception of the display signal and of the output signal and comparison of the two signals with one another; and
outputting of a warning signal if a lack of agreement is present between an output carried out and a visual output to be carried out according to the display signal.

The process according to the additional aspect of the present invention makes it especially advantageously possible to use a separate device for displaying data of an electromedical device. Furthermore, the display of data of different electromedical devices by a single display medium is made possible. An especially great clarity of the medical data can be ensured hereby.

Further, the process advantageously makes possible a continuous checking of the output of data, so that an error in this output can be rapidly detected. As a result, the process according to the present invention for displaying data is especially reliable and is especially only slightly prone to error.

Furthermore, the process according to the present invention makes it possible to detect an overlap if data of another signal, for example, of an administrative display signal, are displayed by the display unit in addition to the display signal and data of the display signal are partially not displayed or are displayed incorrectly as a result.

Preferred embodiments of the process according to the present invention will be described below.

In an advantageous embodiment of the process according to the present invention, the generation of the display signal is carried out such that a corresponding indication on the visual output is displayed if it is not possible to visually output all data received from the electromedical device based on the dimensioning. A user of the process is informed hereby that more data are currently received than can be simultaneously outputted.

In another preferred embodiment, the process further has the following steps:
Outputting of a visual signature changing in time intervals via a signature signal;
reception of the signature signal and outputting of the current visual signature via the display signal; and
comparison of the currently displayed visual signature from the output signal with the current visual signature and provision of checking information as a function of this comparison.

Due to the continuous comparison of the currently displayed visual signature with the visual signature to be currently displayed, an error in the display of data is detected especially rapidly. This is especially advantageous when earlier data continue to be displayed, without newly received data being taken into consideration in this display. In case of such an error, there would be a risk without the checking by means of a visual signature that an error is detected only late during the output.

In an especially preferred embodiment, the process is configured for displaying an output of a plurality of electromedical devices. In this embodiment, the process comprises the following additional step:
reception of data that pertain to at least one additional electromedical device, wherein the visualization type assigned to the data comprises device information that indicates the electromedical device corresponding to the data.

The fact that the process according to the present invention can be carried out independently from the respective electromedical device is advantageously utilized in this embodiment. As a result, it is especially possible that data from the plurality of electromedical devices can be displayed simultaneously by the process according to the present invention. A relevance level of the received data can be inferred due to the respective device information.

Further, the above-mentioned object is accomplished by a program with a program code for carrying out the process according to at least one of the above-described embodiments of the process according to the present invention when the program code is run on a computer, on a processor or on a programmable hardware component.

The program may also carry out only a part of the data processing according to the present invention. At least the function of the display signal unit and of the display signal monitoring unit is preferably controlled by a program and/or by mutually coordinated parts of a program. The program according to the present invention is preferably carried out by a processor of the display system. As an alternative, the program is carried out by a first processor of the display signal unit and by a second processor of the display signal monitoring unit.

The present invention shall now be explained in more detail on the basis of advantageous exemplary embodiments shown schematically in the figures.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of a display system according to the present invention;

FIG. 2 is a schematic view of a second exemplary embodiment of the display system according to the present invention;

FIG. 3 is a schematic view of a third exemplary embodiment of the display system according to the present invention;

FIG. 4 is a schematic view of a first exemplary embodiment of a visual output of the display system according to the present invention;

FIG. 5 is a schematic view of the visual output of the display system according to the present invention for the display of an alarm state;

FIG. 6 is a schematic view of the visual output of the display system according to the present invention for the display of an incomplete output of received data;

FIG. 7 is a schematic view of the visual output of the display system according to the present invention for an overlap of the visual output by an administrative display signal; and

FIG. 8 is a flow chart of a first exemplary embodiment of a process according to the present invention.

### DESCRIPTION OF PREFERRED EMBODIMENTS

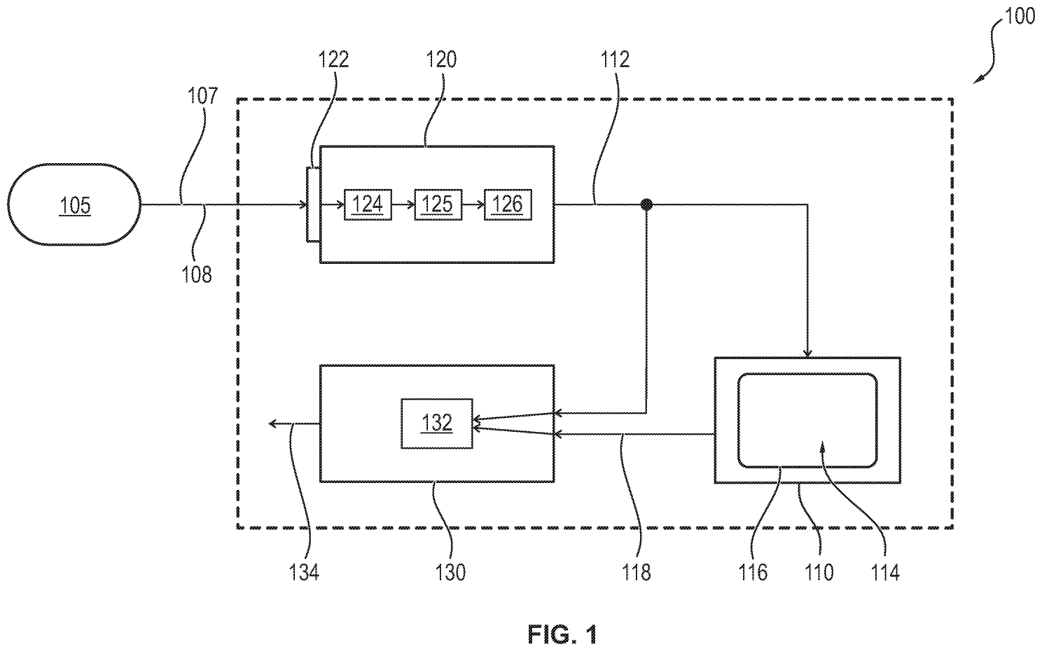

Referring to the drawings, FIG. 1 shows a schematic view of a first exemplary embodiment of a display system 100 according to the present invention.

The display system 100 is configured for displaying an output of at least one electromedical device 105. The display system 100 comprises for this purpose a display unit 110, a display signal unit 120 and a display signal monitoring unit 130.

The display unit 110 is configured to receive a display signal 112 and to display a visual output 114 based on the display signal 112. The visual output 114 is carried out on a display 116 of the display unit 110.

The display signal unit 120 has a communication interface 122 for communication with the electromedical device 105 and is configured to receive data 107, which pertain to the electromedical device 105, via the communication interface 122. The communication interface 122 receives for this purpose the device signal 108 indicating the data 107. The device signal 108 is received in this case in a cable-based manner via an Ethernet connection. In one exemplary embodiment, not shown, the device signal is received via a wireless connection, especially via WLAN, Bluetooth, BLE or ZigBee. In another exemplary embodiment, not shown, the cable-based connection is formed by a bus system, e.g., by a USB system.

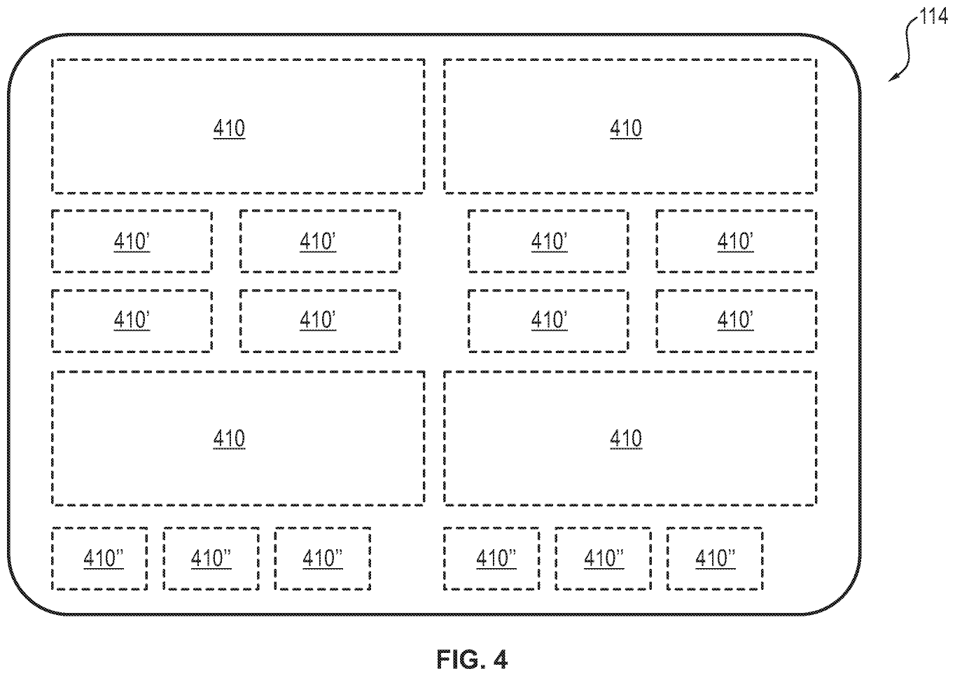

Within the framework of a first processing step 124, the display signal unit 120 is configured to assign the data 107 to a visualization type. In a next, second processing step 125, a respective display type of the received data 107 is determined dynamically as a function of the visualization types to be currently displayed. Based on the data 107 and the respective determined display type, the display signal 112 is generated in a next, third processing step 126 and is outputted to the display unit 110. The determined display type of data from a predefined subset of especially relevant data types comprises at least as a display property the specification that a display is carried out as a visual output visible to a user. This means that an output is also carried out for this predefined subset if it is not currently possible to output all data. Hence, the display of this predefined subset has an especially high priority, so that the display thereof is ensured by the display signal unit 120. Furthermore, the determined display type comprises basically a display position as a display property. The display position describes the position of the display of the corresponding data within the framework of the visual output of the display unit 110. Examples of the display position and of the display property are described within the framework of FIGS. 4 through 6.

The outputted display signal 112 is outputted according to the present invention both to the display unit 110 and to the display signal monitoring unit 130. The display signal 112 outputted to the display unit 110 and that outputted to the display signal monitoring unit 130 may differ from one another, but both display signals comprise according to the present invention the received data and the determined display type as signal components that correspond to each other.

The display signal monitoring unit 130 is connected for signal technology to the display unit 110 and to the display signal unit 120. This connection is a cable-based connection here. The display signal monitoring unit 130 and the display unit 110 preferably have a jointly used memory area. In one exemplary embodiment, not shown, the display signal is transmitted via a wireless connection.

The display signal monitoring unit 130 is configured to receive the display signal from the display signal unit 120 and to receive an output signal 118 from the display unit 110. The display signal 118 contains pieces of information on the carried-out visual output 114 of the display unit 110. As a result, the display signal 118 makes it possible to carry out an analysis to determine how the received and processed data 107 were displayed within the framework of the visual output 114. The display signal monitoring unit 130 is configured here to carry out a comparison between the display signal 112 and the output signal 118 in the monitoring step 132 based on this analysis of the visual output 114 and of the visual output to be carried out. In case of a lack of agreement between the visual output 114 carried out and the visual output to be carried out, the display signal monitoring unit 130 is further configured to output a warning signal 134.

The warning signal 134 is outputted in this case to a user of the display system 100 as an optical signal. In addition or as an alternative, the warning signal is outputted in an exemplary embodiment to the display signal unit. As a result, the display signal unit can carry out a correction of the display signal in an automated manner.

The predefined subset of especially relevant data comprises at least pieces of alarm information and/or clinical measured values of the at least one connected electromedical device. It is ensured hereby that an alarm is always displayed, even if it is not currently possible to display all received data on the display 116. It is thus ensured that a user of the display system 100 is informed of an alarm.

The comparison of visual output 114 carried out and visual output to be carried out also comprises according to the present invention a checking by the display signal monitoring unit 130 to determine whether all letters, numbers and/or symbols to be displayed are visible to the user. It is thus detected whether a font type used is currently unsuitable for the display of the data, whether data are completely or partially overlapped by an output of other data and/or whether a font color is currently unsuitable for the display of the data.

The data 107 received are preferably physiological parameters, which were determined by the at least one electromedical device 105, and/or a setting of the electromedical device 105 and/or an alarm, which was determined by the electromedical device 105. The determined physiological parameters may be, for example, measured values, e.g., invasive or non-invasive blood pressure, heart rate, pulse rate, oxygen saturation, body temperature and/or arrhythmia episodes. The settings of the electromedical device 105 may be, for example, an alarm limit, a mode of operation, a detected alarm state and/or a state of signaling state.

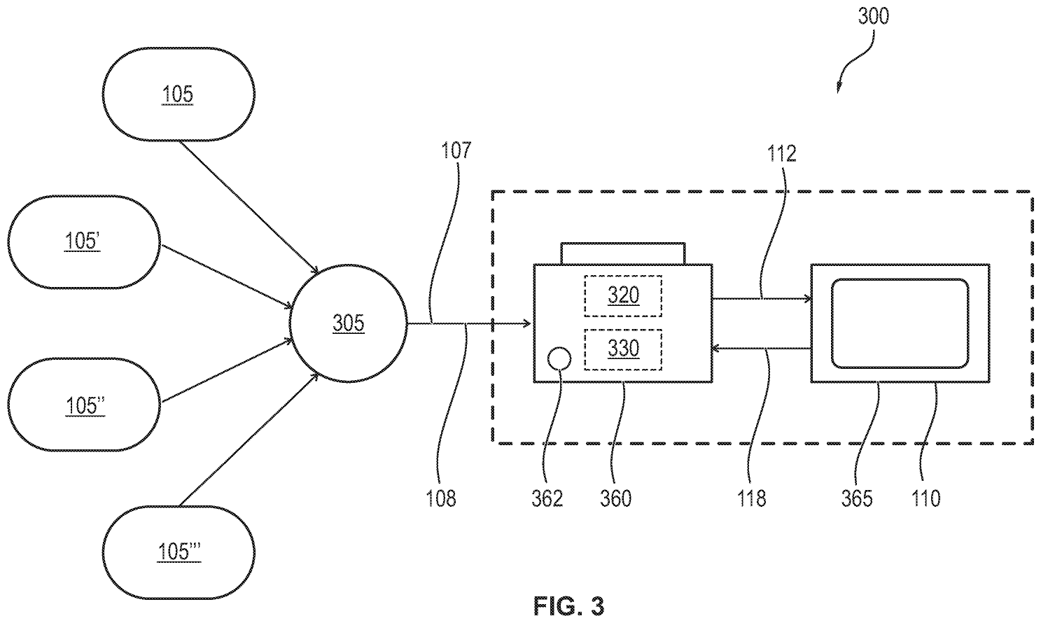

All units 110, 120, 130 of the display system 100 in the exemplary embodiment shown are arranged in a common housing. These units are arranged in separate housings in other exemplary embodiments, as it is shown within the framework of FIG. 3.

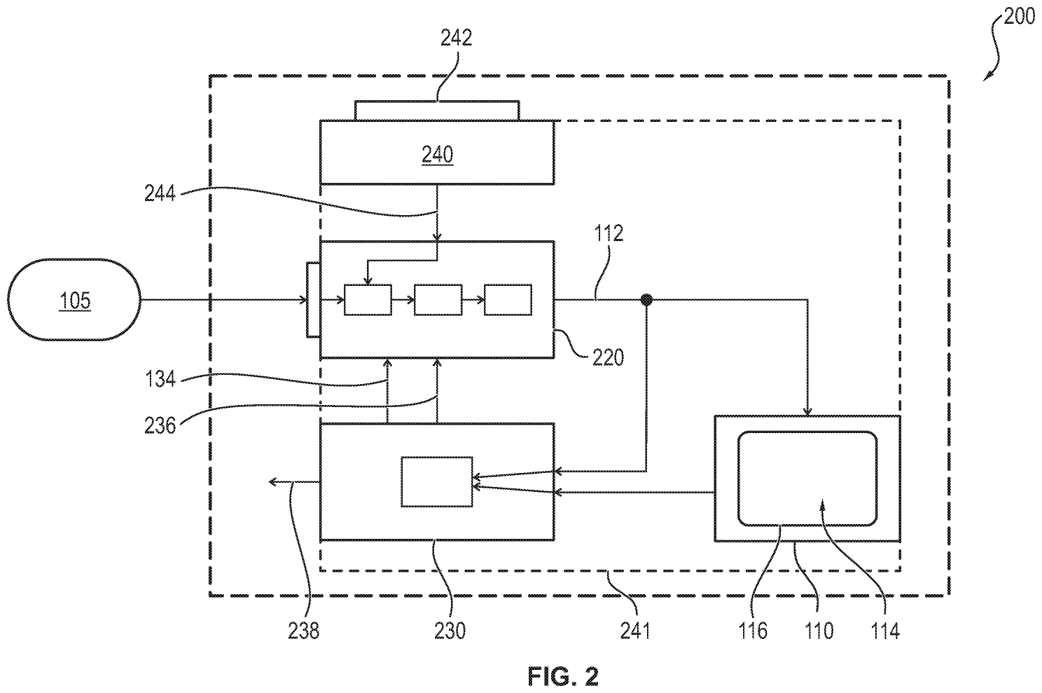

FIG. 2 shows a schematic view of a second exemplary embodiment of the display system 200 according to the present invention.

The display system 200 shown differs from the display system 100 shown in FIG. 1 in that it has an input unit 240.

The input unit 240 is connected for signal technology to the display signal unit 220. It is a cable-based connection in this case. In one exemplary embodiment, not shown, it is a wireless connection. The input unit 240 has, furthermore, a user interface 242, via which a user input of a user of the display system 200 can be received. The user interface 242 is a keyboard in this case. In one exemplary embodiment, not shown, the user interface is a touch operating element, a computer mouse or a joystick. The input unit 240 is configured here to output an input signal 244 depending on the user input.

The display signal unit 220 is configured in the above-described exemplary embodiment to receive the input signal 244 directly via the cable-based connection and to determine a corresponding display type depending on the visualization type assigned to the input signal 244. Data indicated by the user input are outputted via the display signal 112 corresponding to the determined display type and are displayed within the framework of the visual output 114.

The user input may comprise in this case, for example, a configuration of the at least one connected electromedical device 105, for example, a configuration of data of a clinical file or a configuration of data of the electromedical device 105.

Furthermore, the display system 200 differs from the display system 100 shown in FIG. 1 in that the display signal monitoring unit 230 is configured to output the warning signal 134 to the display signal unit 220. The display signal unit 220 is further configured here to receive the warning signal 134 and to correct an error shown therein in an automated manner. This may be carried out, for example, by an automated change of a display property and hence of the determined display type. For example, an automated change of a font color may be necessary for a better visual detectability of data. In another example, an adaptation of the font is necessary to make it possible to distinguish different letters, numbers and/or symbols from one another.

Furthermore, the display system 200 is configured additionally to this to carry out a checking of its own ability to function by the use of a visual signature. The display signal monitoring unit 230 is configured for this to output the predefined visual signature changing at regular time intervals via a signature signal 236 to the display signal unit 220. The predefined visual signature is preferably a combination of letters, numbers and/or symbols. The display signal unit 220 is further configured to output the currently received visual signature via the display signal 112. The display unit 110 will display the visual signature within the framework of the visual output 114. The display signal monitoring unit 230 can therefore compare during the regular operation the displayed visual signature with the signature to be currently displayed and provide checking information depending one this comparison. This is carried out here by the output of a test signal 238.

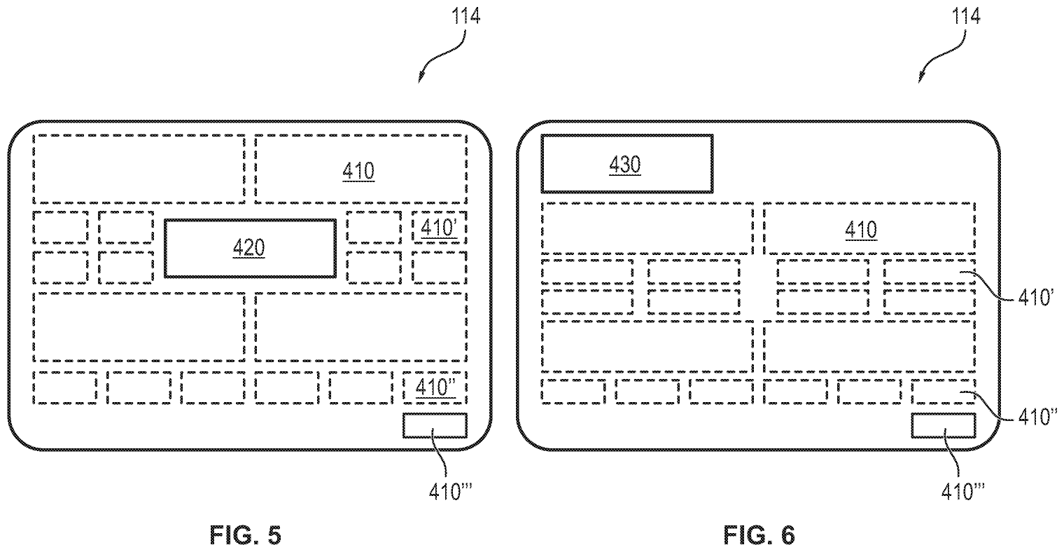

An example of the display of the visual signature is shown within the framework of FIGS. 5 and 6.

The input unit 240 is arranged in a global housing 241 of the display system 200 in the exemplary embodiment shown such that the user interface 242 is arranged at the housing and the housing encloses the display 116.

FIG. 3 shows a schematic view of a third exemplary embodiment of the display system 300 according to the present invention.

The display system 300 differs from the display systems 100 and 200 shown in FIGS. 1 and 2 in that it is configured to display the output of four different electromedical devices 105, 105′, 105″, 105″. The four different electromedical devices 105, 105′, 105″, 105′″ are configured to output their data to a communication network 350. This takes place in this case through a respective wireless connection each, for example, via WLAN, Bluetooth, BLE or ZigBee. In the exemplary embodiment shown, the communication network comprises all the electromedical devices that are used for the treatment of a single patient. In an alternative exemplary embodiment, not shown, the communication network comprises all the electromedical devices that are used for the measurement of a certain physiological parameter or parameter set in a hospital ward in order to obtained thereby an especially good overview about the corresponding physiological state of the patients of that ward.

The display signal unit 320 is configured here to receive the device signal 108, which comprises the data 107, directly from the communication network 350.

The display system 300 is configured in the exemplary embodiment in exactly the same manner as the display system 200 from FIG. 2, the only difference being that the display signal unit 320 and the display signal monitoring unit 330 are arranged in a common housing 360, which is configured at a spaced location from a display housing 365 of the display unit 110. The two modules of the display system 300, which are obtained as a result, have a cable-based connection to one another, via which the display signal 112 and the output signal 118 are transmitted. The common housing 360 has the user interface 242, which is arranged at the housing and which is again a keyboard in this case. Furthermore, an optical output element, especially an LED, which provides an optical output 362 depending on the determined checking information and hence on the test signal, is arranged in the common housing 360. The LED is not on in this case if the current visual signature agrees with the outputted visual signature, and the LED is on, preferably with a red color, if the current visual signature does not agree with the outputted visual signature.

FIG. 4 shows a schematic view of a first exemplary embodiment of a visual output 114 of a display system 300 according to the present invention.

The visual output 114 has a plurality of output areas 410, 410′, 410″ indicated by broken lines. The concrete data-specific contents of the output areas 410, 410′, 410″ were not shown for reasons of clarity. The representation of broken or closed frames, which will hereinafter be used, serves only the purpose of ensuring better visualization of the output areas 410, 410′, 410″, but no frames are preferably visible within the visual output 114 to a user of the display system 300. The output areas differ in their size. In the exemplary embodiment shown, the display unit has received data four times, and these data were assigned to a visualization type, to which a high relevance level is assigned. These four outputs correspond to the four large output areas 410. An example of such a visualization type with high relevance level is a physiological measured value, for example, the oxygen saturation in the blood. Furthermore, data with a medium relevance level were received, and eight output areas 410′ of a medium size were assigned to these data. Finally, data with a low relevance level were obtained as well, and six output areas 410″ of a small size were assigned to them by the display signal unit.

The large output areas 410 are arranged in both the upper area and in the central area of the visual output 114 and can as a result also be detected rapidly based on their position for a user of the display system 300.

The arrangement of the output areas and the selection of the size thereof were determined dynamically based on the visualization types to be currently displayed. In case other visualization types should currently be visualized, the same display system would therefore provide another arrangement of output areas.

The display size and the display position of received data form display properties in this exemplary embodiment. All the display properties combined form the respective display type of received data. It is not shown in FIG. 4 that a font type and a font color of the data to be displayed likewise form a display property.

In one exemplary embodiment according to the present invention, not shown, only the display position and a piece of information on whether a display of the data takes place form a respective display property of the dynamically determined display type.

FIGS. 5 and 6 show a schematic view of the visual output 114 of the display system according to the present invention for the display of an alarm state 420 (FIG. 5) and for the display of an incomplete output 430 of received data (FIG. 6).

The visual outputs 114 shown in FIGS. 5 and 6 are based on the visual output 114 shown in FIG. 4, and they show how this visual output 114 is changed by the display of an alarm state 420 or by the display of an incomplete output 430.

It can be seen that the display of an alarm state 420 leads to a shift and to a reduction in the size of other output areas 410, 410′, 410″. As a result, a user of the display system can detect the presence of the alarm state especially rapidly.

The display of an incomplete output 430 typically takes place in an edge area of the visual output 114, in this case at the upper edge. The display of an incomplete output 430 informs a user of the display system that not all the data received by the display system can be visually outputted by the display system based on the dimensioning of the visual output. This display likewise causes the other output areas 410, 410′, 410″ to be shifted and the sizes thereof to be reduced.

Furthermore, an especially small output area 410′″ is provided in FIGS. 5 and 6 for the visual signature at the lower edge of the visual output 114. The visual signature contained in it is of no interest to a user of the display system and is used only for checking the display signal unit by the display signal monitoring unit, as was described in detail above. In one exemplary embodiment, not shown, a font color, which is similar to a background color being used, is selected, furthermore, for the signature, so that the visual signature is not disturbing for a user of the display system.

The output areas 410, 410′, 410″, 410′″ shall not be considered to represent a limiting selection. Other sizes, positions and shapes of output areas are provided in exemplary embodiments that are not shown. The data may be outputted here within the output areas in the form of letters, numbers, symbols, tables, diagrams and of any other graphic representation.

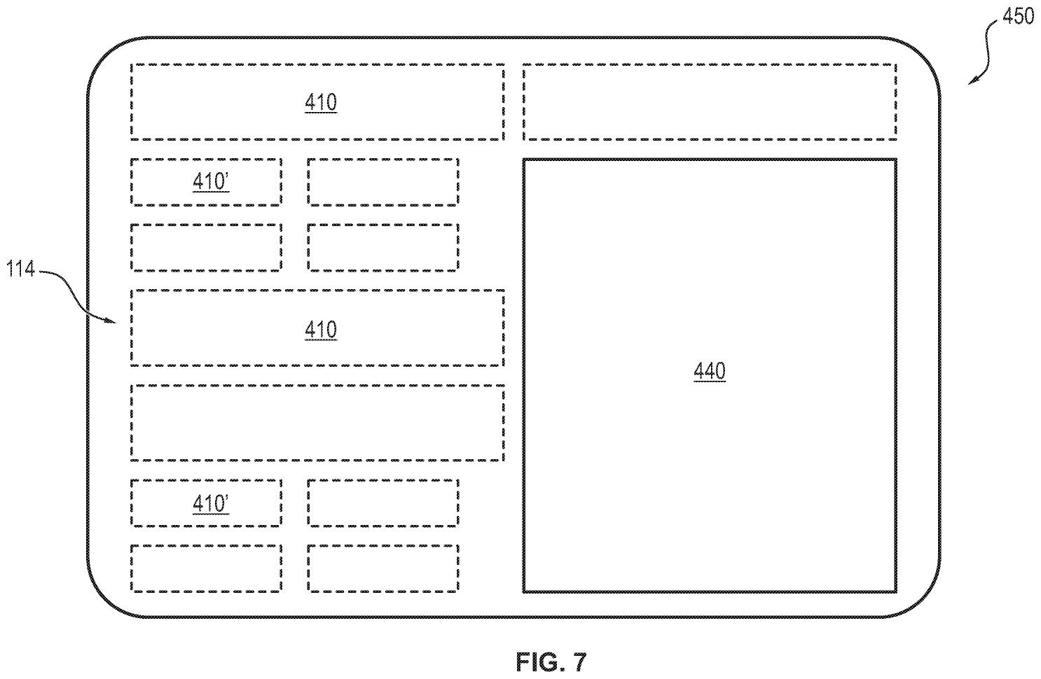

FIG. 7 shows a schematic view of the visual output of the display system according to the present invention for an overlap of the visual output by an additional output 440 triggered by an administrative display signal.

The display unit is configured, furthermore, in the exemplary embodiment shown to receive an additional administrative display signal and to display a combined visual output 450 based on the display signal and the additional administrative display signal, in which case the additional administrative display signal indicates non-medical data of an external device. The combined output 450 comprises here the additional output 440, which is overlapped by the original visual output 114 shown in FIG. 4, and a part of the original visual output 114. The individual output areas 410, 410′, 410″ were adapted by the display signal monitoring unit via a corresponding adaptation of the display signal. This adaptation was carried out such that the four large output areas 410, to which a high relevance level is assigned, continue to be clearly visible and their display position has changed only slightly. Furthermore, the eight output areas 410′ of a medium size, to which a medium relevance level is assigned, are configured such that their size is slightly reduced compared to the original output 114 shown in FIG. 4 and their display position is changed. The output areas of a small size, which are shown in FIG. 4, and to which only a low relevance level is assigned, were removed from the combined output 450 because of the additional output 440.

FIG. 7 illustrates how a combined output 450 can take the assigned relevance level into consideration in order to display all relevant data in a manner suitable for a user despite an overlap due to the additional output 440, which overlap cannot be influenced.

In one exemplary embodiment, not shown, a visual signature is also outputted by the display unit in addition to the additional output.

In another exemplary embodiment, not shown, it is also displayed, in addition to the additional output, that it was not possible to output all the received data. In the case shown, such a display of an incomplete output is not shown because only data to which a low relevance level is assigned were not displayed. If it had been impossible to display especially relevant data, a corresponding display of an incomplete output would have been displayed.

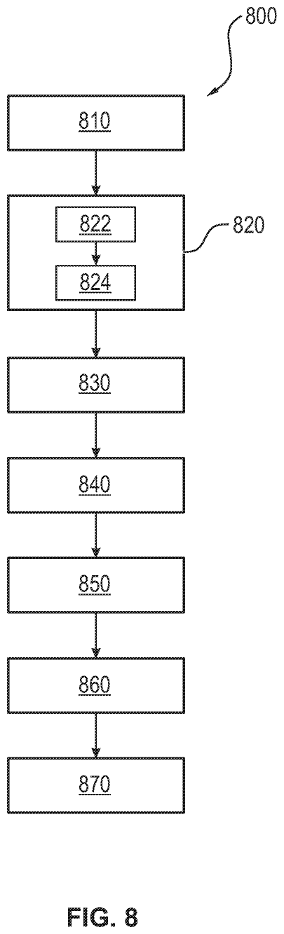

FIG. 8 shows a flow chart of a first exemplary embodiment of a process 800 according to the present invention.

The process 800 according to the present invention for displaying an output of at least one electromedical device comprises a sequence of the steps described below in the order shown.

A first step 810 comprises the reception of data that pertain to the at least one electromedical device.

A next step 820 comprises in a first partial step 822 an assignment of the received data to a respective visualization type and in a second partial step 824 a dynamic determination of a respective display type of the received data based on the visualization types to be currently displayed.

Another step 830 comprises the generation and outputting of a display signal based on the received data and on the determined display type, wherein the determined display type of data from a predefined subset of especially relevant data types comprises at least as a display property the fact that a display is carried out as a visual output visible to a user and wherein the determined display type further comprises a display position as a display property.

A next step 840 comprises the reception of the display signal and the display of a visual output based on the display signal.

Another step 850 comprises the transmission of an output signal, which contains information on the visual output carried out.

A next step 860 comprises the reception of the display signal and of the output signal and a comparison of the two signals with one another.

A last step 870 comprises the outputting of a warning signal if there is a lack of agreement between the visual output carried out and a visual output to be carried out according to the display signal.

Steps 810 through 860 are carried out one after another. This is carried out preferably at regular time intervals, which are selected such that the visual output takes place nearly in real time with the reception of the data. Step 870 is not carried out regularly, because it is carried out only in the case of an error, namely, in case of a lack of agreement between a visual output carried out and a visual output to be carried out.

To make it possible to display a current state of the electromedical device, the regular time intervals between the respective output of the display signal to the display unit are longer than 1 sec, preferably longer than 2 sec and especially preferably longer than 3 sec in one exemplary embodiment, not shown. It can be ensured hereby that an individual display signal is not generated for each received signal, but a group of signals that belong physiologically together is processed within the framework of a single display signal.

In another exemplary embodiment, not shown, the reception of data triggers the displayed process 800 directly. A visual output of the received data can take place as a result nearly in real time.

While specific embodiments of the invention have been shown and described in detail to illustrate the application of the principles of the invention, it will be understood that the invention may be embodied otherwise without departing from such principles.

### LIST OF REFERENCE NUMBERS

100, 200, 300 Display system
105, 105′, 105″, 105′″ Electromedical device
107 Data
108 Device signal
110 Display unit
112 Display signal
114 Visual output
116 Display
118 Output signal
120, 220, 320 Display signal unit
122 Communication interface
124 First processing step
125 Second processing step
126 Third processing step
130, 230, 330 Display signal monitoring unit
132 Monitoring step
134 Warning signal
236 Signature signal
238 Test signal
240 Input unit
241 Global housing
242 User interface
244 Input signal
350 Communication network
360 Common housing
362 Optical output
365 Display housing
410, 410′, 410″, 410′″ Output areas
420 Display of an alarm state
430 Display of an incomplete output
440 Additional output
450 Combined output
800 Process
810, 820, 830, 840, Process steps
850, 860, 870
822, 824 Partial steps of process step 720

What is claimed is:

1. An electromedical device display system for displaying an output of an electromedical device, the display system comprising:
a display unit configured to receive a display signal and to display a visual output on the basis of the display signal;
a display signal unit comprising a communication interface for communication with the electromedical device, the display signal unit being configured to receive electromedical device data, which pertain to the electromedical device, via the communication interface, to assign the data to a visualization type, to dynamically determine a respective display type of the received data as a function of visualization types currently to be displayed and to generate the display signal based on the data and based on the respective determined display type and to output the display signal to the display unit, wherein the determined display type of data is from a predefined subset of relevant data types that comprises a display property that indicates that a display is carried out as a visual output visible to a user, and a display property that indicates a display position; and
a display signal monitoring unit signal connected to the display unit and to the display signal unit, the display signal monitoring unit being configured to receive the display signal, to receive an output signal from the display unit, wherein the output signal contains information on a visual output carried out, to compare the display signal and the output signal with one another and to output a warning signal in case of a lack of agreement between the visual output carried out and the visual output to be carried out according to the display signal.

2. An electromedical device display system in accordance with claim 1, wherein the display signal monitoring unit is configured to output the warning signal to the display signal unit.

3. An electromedical device display system in accordance with claim 1, wherein the predefined subset of relevant data types comprises alarm information and/or clinical measured values.

4. An electromedical device display system in accordance with claim 1, wherein the display signal unit is further configured to generate the display signal such that a corresponding indication is displayed via the visual output if it is not possible to visually output all the data received from the electromedical device by the display unit.

5. An electromedical device display system in accordance with claim 1, wherein the display signal unit is further configured to determine a display size and/or the display position of received data of a visualization type depending on a relevance level assigned to this visualization type.

6. An electromedical device display system in accordance with claim 1, wherein:
the display system further has an input unit, which is signal connected to the display signal unit and has a user interface;
the input unit is configured to output an input signal via the user interface depending on a user input of a user of the display system, and
the display signal unit is further configured to receive the input signal and to determine a corresponding display type depending on the visualization type assigned to the input signal and to generate and to output the display signal based on all currently received data and on the respective determined display type.

7. An electromedical device display system in accordance with claim 1, wherein:
the display signal monitoring unit is further configured to output a visual signature changing at time intervals via a signature signal to the display signal unit;
the display signal unit is further configured to output a current visual signature via the display signal; and
the display signal monitoring unit is further configured to compare via the display signal the currently displayed visual signature with the current visual signature and to provide test information depending on this comparison.

8. An electromedical device display system in accordance with claim 1, wherein the display unit is further configured to receive an additional administrative display signal and to display a combined visual output based on the display signal and the additional administrative display signal, wherein the additional administrative display signal indicates non-medical data of an external device.

9. An electromedical device display system in accordance with claim 1, wherein the communication interface is configured to receive data from a plurality of electromedical devices, wherein the visualization type assigned to the data by the display signal unit comprises device information, which indicates the electromedical device corresponding to the received data.

10. A process for displaying an output of an electromedical device, the process comprising the steps of:
receiving data, which pertain to the electromedical device;
assigning the received data to a respective visualization type and dynamically determining a respective display type of the received data based on the visualization types to be currently displayed;
generating and outputting a display signal based on the received data and based on the respective determined display type, wherein the determined display type of data is from a predefined subset of relevant data types comprising a display property that indicates that a display is carried out as a visual output visible to a user, and a display property that indicates a display position;
receiving the display signal and displaying a visual output based on the display signal;
transmitting an output signal, which contains information on the visual output carried out;
receiving the display signal and the output signal and comparing the display signal and the output signal with one another; and
outputting a warning signal if there is a lack of agreement between the visual output carried out and a visual output to be carried out according to the display signal.

11. A process in accordance with claim 10, wherein the generation of the display signal is carried out such that a corresponding indication on the visual output is displayed if it is not possible to visually output all the data received from the electromedical device based on the dimensioning of the visual output.

12. A process in accordance with claim 10, further comprising:
outputting a visual signature changing at time intervals via a signature signal;
receiving the signature signal and outputting of the current visual signature via the display signal; and
comparing the currently displayed visual signature from the output signal with the current visual signature and provide a test signal as a function of the comparison.

13. A process in accordance with claim 10, wherein the process is configured for displaying an output of a plurality of electromedical devices, the process further comprising the step of:
receiving data that pertain at least to one additional electromedical device, wherein the visualization type assigned to the data comprises device information, which indicates the electromedical device corresponding to the received data.

14. A process according to claim 13, further comprising providing a program with a program code for carrying out the process when the program code is run on a computer, on a processor or on a programmable hardware component.
